(19) United States
(12) Patent Application Publication
(10) Pub. No.: US 2005/0028249 A1
(43) Pub. Date: Feb. 10, 2005

(54) Tanning cap

(76) Inventor: Christine Marie Molnar, Grosse Ile, MI (US)

Correspondence Address: Christine Marie Molnar, 9365 Highland, Grosse Ile, MI, 48138, US

(21) Appl. No.: 10/894,814

(22) Filed: Jul. 20, 2004

Related U.S. Application Data
(60) Provisional application No. 60/488,823, filed on Jul. 22, 2003.

(51) Int. Cl. A42B 1/00; A61F 9/00

(52) U.S. Cl. 2/200.1

(57) ABSTRACT

A tanning cap for protecting the hair of a wearer from damage due to exposure to ultraviolet light emitted from a tanning apparatus. The tanning cap is a hair covering consisting of at least one layer, with an interior cavity or inner hollow space, and having an adjustable opening which allows the head of a wearer to access the interior cavity or inner hollow space. The layer(s) may be crafted from material(s) that is(are): reflective; insulating; screening; diffusing; breathable; or a combination of any such materials. The size of the tanning cap opening which allows a wearer's head to access the interior cavity or inner hollow space is adjustable. The tanning cap opening consists of a “band” and a closure mechanism. The size of the tanning cap opening adjusts as a result of adjustment of the closure mechanism along the band. The “band” encompasses the full peripheral edge, or circumference, of the cap; creating a non-stretchable, non-elastic peripheral edge. The “band” being created from stitching along the full peripheral edge. The closure mechanism is comprised of two pieces, or tabs, being of hook and loop arrangements and/or hook and loop strips and/or being comprised of a drawstring mechanism. An adjustable tanning cap opening allows the cap to transform to fit the head of the wearer thereby offering a one-size-fits-all approach.

### APPL NO.

Provisional Patent Application No. 60/488,823

### FILED

Provisional Patent Application filed Jul. 22, 2003

### DRAWING SHEETS

one (1)

### CLAIMS

twelve (12)

### REFERENCES CITED

3,766,565 A 10/1973 Cozzens - - - 2/200

5,481,759 A 1/1996 Rinaldi - - - 2/195.1

6,216,268 B1 4/2001 Nguyen et al. - - - 2/174

6,349,415 A 2/2002 Gong - - - 2/195.5

6,625,817 B2 9/2003 Wasmuth - - - 2/174

### SUMMARY OF THE INVENTION

The present invention provides a protective hair covering that adjusts to fit snuggly on the head of a wearer or adjusts to whatever preference of fit is selected by a wearer. The present invention is reusable. It may also be reflective, insulating, screening, diffusing, breathable, or a combination of any of the aforementioned.

### FIELD OF THE INVENTION

The present invention generally relates to a hair covering that is intended to be worn by individuals using any variety of tanning apparatus (bed, booth, standup, etc.).

### BACKGROUND OF THE INVENTION

It is common for individuals to use any variety of tanning apparatus to maintain a darkened skin tone. The ultraviolet light emitted by any variety of tanning apparatus may, and has been known to, cause damage to a user's hair.

### BRIEF DESCRIPTION OF THE DRAWINGS

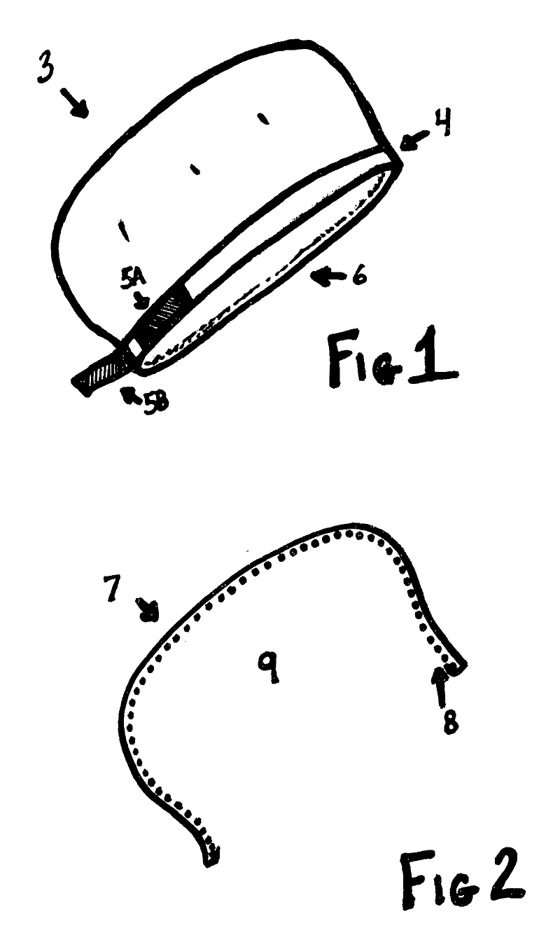

One (1) drawing sheet is attached, containing two figures. FIG. 1 and FIG. 2.

FIG. 1 shows a preferred embodiment of the present invention. With reference to FIG. 1: item 3 shows the exterior portion of the supple, flexible body of the cap; item 4 shows the “band” portion of the cap which is created from stitching along the full peripheral edge, or circumference, of the cap thereby creating a non-stretchable, non-elastic peripheral edge;

item 5 shows a closure mechanism, in FIG. 1 the closure mechanism is depicted as a closure mechanism comprised of two pieces, or tabs, being of hook and loop arrangements and/or hook and loop strips;

item 5A indicates the longer, fully secured tab;

item 5B indicates the shorter, hinged tab;

item 6 identifies the tanning cap opening which allows a wearer's head to access the interior cavity or inner hollow space

FIG. 2 is a cut-away view of a preferred embodiment of the present invention.

With reference to FIG. 2:

item 7 shows a cut-away view of the exterior portion of the supple, flexible body of the cap; item 8 (dotted line) indicates the location of interior and/or intermediate layer(s) of the supple, flexible body of the cap:

item 9 shows the interior cavity or inner hollow space

### DETAILED DESCRIPTION OF THE PREFERRED EMBODIMENT

The tanning cap is a hair covering consisting of at least one layer, with an interior cavity or inner hollow space, and having an adjustable opening which allows the head of a wearer to access the interior cavity or inner hollow space. FIGS. 1 & 2 and items 6 & 9.

The layer(s) may be crafted from material(s) that is(are): reflective; insulating; screening; diffusing; breathable; and/or a combination of any such materials.

The size of the tanning cap opening which allows a wearer's head to access the interior cavity or inner hollow space is adjustable. An adjustable tanning cap opening allows the cap to transform to fit the head of the wearer thereby offering a one-size-fits-all approach.

The tanning cap opening consists of a “band” and a closure mechanism. FIG. 1, items 4 & 5. The size of the tanning cap opening adjusts as a result of adjustment of the closure mechanism along the band. The “band” encompasses the full peripheral edge, or circumference, of the cap; creating a non-stretchable, non-elastic peripheral edge. The “band” being created from stitching along the full peripheral edge of the cap. FIG. 1, item 4.

The closure mechanism may be comprised of two pieces, or tabs, being of hook and loop arrangements and/or hook and loop strips (for example: as distributed under the trademark VELCRO). The coacting elements of the hook-and-pile type materials are disposed along their respective tabs to be in registration with one another. FIG. 1, item 5.

The two flexible pieces or tabs each being secured by stitching, gluing, etc., to the exterior surface of the tanning cap opening's band. One of the pieces is shorter (smaller in size, shorter in length) than the other piece. The longer piece is secured in its entirety to the exterior surface of the tanning cap opening's band. FIG. 1, item 5A.

The shorter piece is secured to the exterior of the band by sewing, gluing, etc, a short distance from the longer piece. Only one end of the shorter piece is secured to the exterior surface of the tanning cap band so that the point of attachment of the shorter piece to the exterior surface serves as a hinge; thereby allowing the free portion of the hinged piece to fasten at varying points along the longer piece as desired by the wearer. FIG. 1, item 5B.

Other closure mechanisms may be employed instead of hook and loop arrangements. For example, the closure mechanism may be comprised of a drawstring mechanism. The drawstring being located along the band that encompasses the full peripheral edge, or circumference, of the cap wherein the drawstring was affixed within the band area during construction of the band. FIG. 1, item 4.

An adjustable closure mechanism allows the tanning cap to adjust to fit snugly around the head of a wearer.

This embodiment intends to include variations in:

1) tab lengths and placement along the exterior surface of the tanning cap band;

2) the type of closure mechanisms employed to achieve an adjustable cap (hook & loop, snaps, buttons, hook & eye, drawstring, pull-cord);

3) the shape of the “dome” or “skull” portion of the tanning cap; and

4) the types of materials used to create any aspect of the tanning cap: layers; closures; band; seams; stitching

Failure to specifically identify potential, possible, obvious, discoverable variations does not signify an intention by the inventor to preclude protection under this section.

1) A tanning cap consisting of:
a) a supple, flexible layered body having an exterior layer, intermediate layer(s) and an interior layer
1. the exterior layer being of a reflective, screening or diffusing material having an ample Ultra Violet Protection Factor rating to reflect, screen or diffuse ultraviolet rays (for example: SolarMax as trademarked by Dupont);
2. the intermediate layer(s) being of a reflecting, screening, insulating or diffusing material having an ample Ultra Violet Protection Factor rating to reflect, screen or diffuse ultraviolet rays (for example: SolarMax as trademarked by Dupont); and
b) the supple, flexible layered body forming an interior cavity or hollow space
1. the interior layer being of an insulating material for reducing heat build up within the interior cavity during use; and
c) having an opening which allows the head of a wearer to access the interior cavity
1. the tanning cap opening being adjustable and consisting of a “band” being created from stitching along the full peripheral edge and encompassing the full peripheral edge, or circumference, of the cap thereby creating a non-stretchable, non-elastic peripheral edge and having a closure mechanism to allow the wearer to adjust the fit of the tanning cap

2) The tanning cap as described in claim 1 wherein the cap consists of:
a) a supple, flexible layered body having an exterior layer, intermediate layer(s) and an interior layer
1. the exterior layer being of a reflective, screening or diffusing material having an ample Ultra Violet Protection Factor rating to reflect, screen or diffuse ultraviolet rays (for example: SolarMax as trademarked by Dupont);
2. the intermediate layer(s) being of an insulating material for reducing heat build up within the interior cavity during use; and
3. the interior layer being of an insulating material for reducing heat build up within the interior cavity during use

3) The tanning cap as described in claim 1 wherein the cap consists of:
a) a supple, flexible layered body having an exterior layer, intermediate layer(s) and an interior layer
1. the exterior layer being of a reflective, screening or diffusing material having an ample Ultra Violet Protection Factor rating to reflect, screen or diffuse ultraviolet rays (for example: SolarMax as trademarked by Dupont);
2. the intermediate layer(s) being of a reflecting, screening, insulating or diffusing material having an ample Ultra Violet Protection Factor rating to reflect, screen or diffuse ultraviolet rays (for example: SolarMax as trademarked by Dupont); and
3. the interior layer being of a reflecting, screening, insulating or diffusing material having an ample Ultra Violet Protection Factor rating to reflect, screen or diffuse ultraviolet rays (for example: SolarMax as trademarked by Dupont)

4) The tanning cap as described in claim 1 wherein the cap consists of:
a) a supple, flexible layered body having an exterior layer, intermediate layer(s) and an interior layer wherein the exterior layer is of a breathable material
1. the exterior layer also being of a reflective, screening or diffusing material having an ample Ultra Violet Protection Factor rating to reflect, screen or diffuse ultraviolet rays (for example: SolarMax as trademarked by Dupont);
2. the intermediate layer(s) being of an insulating material for reducing heat build up within the interior cavity during use; and
3. the interior layer being of an insulating material for reducing heat build up within the interior cavity during use

5) The tanning cap as described in claim 1 wherein the cap consists of:
a) a supple, flexible layered body having an exterior layer, intermediate layer(s) and an interior layer wherein the exterior layer and intermediate layer(s) are of a breathable material
1. the exterior layer also being of a reflective, screening or diffusing material having an ample Ultra Violet Protection Factor rating to reflect, screen or diffuse ultraviolet rays (for example: SolarMax as trademarked by Dupont);
2. the intermediate layer(s) also being of a reflective, screening or diffusing material having an ample Ultra Violet Protection Factor rating to reflect, screen or diffuse ultraviolet rays (for example: SolarMax as trademarked by Dupont); and
3. the interior layer being of an insulating material for reducing heat build up within the interior cavity during use

6) The tanning cap as described in claim 1 wherein the cap consists of:
a) a supple, flexible layered body having an exterior layer, intermediate layer(s) and an interior layer wherein the exterior layer, the intermediate layer(s) and the interior layer are all of a breathable material, thereby permitting heat to escape without any non-breathable layer(s) to impede the escape of heat
1. the exterior layer also being of a reflective, screening or diffusing material having an ample Ultra Violet Protection Factor rating to reflect, screen or diffuse ultraviolet rays (for example: SolarMax as trademarked by Dupont);
2. the intermediate layer(s) also being of a reflecting, screening, insulating or diffusing material having an ample Ultra Violet Protection Factor rating to reflect, screen or diffuse ultraviolet rays (for example: SolarMax as trademarked by Dupont); and
3. the interior layer also being of a reflecting, screening, insulating or diffusing material having an ample Ultra Violet Protection Factor rating to reflect, screen or diffuse ultraviolet rays (for example: SolarMax as trademarked by Dupont)

7) A tanning cap consisting of:
a) a supple, flexible layered body consisting only of an exterior layer and an interior layer without any intermediate layer(s) between the exterior layer and the interior layer
1. the exterior layer being of a reflective, screening, insulating or diffusing material having all ample Ultra Violet Protection Factor rating to reflect, screen, insulate or diffuse ultraviolet rays (for example: SolarMax as trademarked by Dupont);
b) the supple, flexible layered body defining an interior cavity or hollow space
1. the interior layer being of an insulating material for reducing heat build up within the interior cavity during use; and
c) having an opening which allows the head of a wearer to access the interior cavity
1. the tanning cap opening being adjustable and consisting of a “band” being created from stitching along the full peripheral edge and encompassing the full peripheral edge, or circumference, of the cap thereby creating a non-stretchable, non-elastic peripheral edge and having a closure mechanism to allow the wearer to adjust the fit of the tanning cap

8) The tanning cap as described in claim 7 wherein the cap consists of:
a) a supple, flexible layered body consisting only of an exterior layer and an interior layer without any intermediate layer(s) between the exterior layer and the interior layer
1. the exterior layer being of a reflective, screening, insulating or diffusing material having an ample Ultra Violet Protection Factor rating to reflect, screen, insulate or diffuse ultraviolet rays (for example: SolarMax as trademarked by Dupont); and
2. the interior layer being of a reflecting, screening, insulating or diffusing material having an ample Ultra Violet Protection Factor rating to reflect, screen or diffuse ultraviolet rays (for example: SolarMax as trademarked by Dupont)

9) The tanning cap as described in claim 7 wherein the cap consists of:
a) a supple, flexible layered body consisting only of an exterior layer and an interior layer without any intermediate layer(s) between the exterior layer and the interior layer
1. the exterior layer being of a breathable material;
2. the exterior layer also being of a reflective, screening, insulating or diffusing material having an ample Ultra Violet Protection Factor rating to reflect, screen, insulate or diffuse ultraviolet rays (for example: SolarMax as trademarked by Dupont); and
2. the interior layer being of a reflecting, screening, insulating or diffusing material having an ample Ultra Violet Protection Factor rating to reflect, screen or diffuse ultraviolet rays (for example: SolarMax as trademarked by Dupont)

10) The tanning cap as described in claim 7 wherein the cap consists of:
a) a supple, flexible layered body consisting only of an exterior layer and an interior layer without any intermediate layer(s) between the exterior layer and the interior layer and wherein the exterior layer and the interior layer are both of a breathable material, thereby permitting heat to escape from the interior cavity through the exterior layer and the interior layer without any non-breathable intermediate layer(s) between the exterior layer and interior layer to impede the escape of heat
1. the exterior layer also being of a reflective, screening, insulating or diffusing material having all ample Ultra Violet Protection Factor rating to reflect, screen, insulate or diffuse ultraviolet rays (for example: SolarMax as trademarked by Dupont); and
2. the interior layer also being of a reflecting, screening, insulating or diffusing material having an ample Ultra Violet Protection Factor rating to reflect, screen or diffuse ultraviolet rays (for example: SolarMax as trademarked by Dupont)

11) A tanning cap consisting of:
a) a supple, flexible body consisting only of an exterior layer and an interior layer without any intermediate layer(s) between the exterior layer and the interior layer;
b) the exterior layer and the interior layer being in direct contact with one another;
c) the exterior layer and the interior layer being comprised of a single layer of material thereby allowing the single layer to serve as both the exterior layer and the interior layer of the tanning cap
1. the single layer serving as both the exterior layer and the interior layer being of a reflective, screening, insulating or diffusing material having an ample Ultra Violet Protection Factor rating to reflect, screen, insulate or diffuse ultraviolet rays (for example: SolarMax as trademarked by Dupont); and
d) the supple, flexible layered body forming an interior cavity or hollow space; and
e) having an opening which allows the head of a wearer to access the interior cavity
1. the tanning cap opening being adjustable and consisting of a “band” being created from stitching along the full peripheral edge and encompassing the full peripheral edge, or circumference, of the cap thereby creating a non-stretchable, non-elastic peripheral edge and having a closure mechanism to allow the wearer to adjust the fit of the tanning cap

12) The tanning cap as described in claim 11 wherein the cap consists of:
a) a supple, flexible body consisting only of an exterior layer and an interior layer without any intermediate layer(s) between the exterior layer and the interior layer with the exterior layer and interior layer being in direct contact with one another and being comprised of a single layer of material thereby allowing a single layer to serve as both the exterior layer and the interior layer of the tanning cap; and
b) the single layer serving as both the exterior layer and the interior layer being of a breathable material, thereby permitting heat to escape from the interior cavity through the single layer serving as both the exterior layer and the interior layer without any non-breathable layer(s) to impede the escape of heat
1. the single layer serving as both the exterior layer and the interior layer also being of a reflective, screening, insulating or diffusing material having an ample Ultra Violet Protection Factor rating to reflect, screen, insulate or diffuse ultraviolet rays (for example: SolarMax as trademarked by Dupont)
